(19) United States
(12) Patent Application Publication
(10) Pub. No.: US 2006/0057092 A1
(43) Pub. Date: Mar. 16, 2006

(54) Cosmetic use of a composition including an ascorbic acid compound and polyamide particles

(75) Inventor: Catherine Marion, Antony (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/224,959

(22) Filed: Sep. 14, 2005

Related U.S. Application Data
(60) Provisional application No. 60/612,875, filed on Sep. 27, 2004.

(30) Foreign Application Priority Data
Sep. 16, 2004 (FR) 04/52074

(51) Int. Cl. A61K 8/73

(52) U.S. Cl. 424/70.13; 424/70.17

(57) ABSTRACT

The present invention relates to the cosmetic use, for the care of greasy skin, of a composition including, in a physiologically acceptable medium, (a) at least one water-soluble ascorbic acid compound and (b) porous polyamide particles having a mean diameter, by volume, of less than or equal to 10 μm, provided that the composition is not a water-in-oil emulsion.

### REFERENCE TO PRIOR APPLICATIONS

This application claims priority to U.S. provisional application 60/612,875 filed Sep. 27, 2004, and to French patent application 0452074 filed Sep. 16, 2004, both incorporated herein by reference.

### FIELD OF THE INVENTION

The present invention relates to the use, for example for the care of greasy skin, of a composition comprising, preferably in a physiologically acceptable medium, (a) at least one water-soluble ascorbic acid compound and (b) porous polyamide particles having a mean diameter, by volume, of less than or equal to 10 μm. Preferably, the composition is not a water-in-oil emulsion.

It also relates to a cosmetic process for caring for greasy skin, in particular for the purpose of mattifying the skin, comprising the topical application to the latter of a composition as defined above.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The shininess of the skin is a problem affecting more particularly adolescents but which can also be displayed during adulthood under the effect in particular of hyperproduction of androgens. A shiny skin is generally a hyperseborrhoeic skin characterized by an excessive secretion and an excessive excretion of sebum, generally resulting in a level of sebum of greater than 200 μg/cm², measured on the forehead.

Sebum is the natural product from the sebaceous gland, which constitutes an appendix of the pilosebaceous unit. It is essentially a more or less complex mixture of lipids. Conventionally, the sebaceous gland produces squalene, triglycerides, aliphatic waxes, cholesterol waxes and possibly free cholesterol (Stewart, M. E., Semin Dermatol, 11, 100-105 (1992)). A variable portion of the triglycerides formed is converted to free fatty acids under the action of bacterial lipases.

The sebocyte is the relevant cell of the sebaceous gland. The production of sebum is associated with a terminal differentiation programme of this cell. During this differentiation, the metabolic activity of the sebocyte is essentially centred on the biosynthesis of the lipids (lipogenesis) and more specifically on fatty acid neosynthesis.

While sebum is normally a natural moisturizer for the epidermis, the excess production of sebum can have unpleasant effects aesthetically (glistening skin, less good retention of makeup, formation of blackheads), indeed even can constitute a favourable terrain for the uncontrolled growth of saprophytic bacterial flora, such as P. acnes, and can bring about acne lesions.

In order to combat hyperseborrhoea, the prior art has thus provided various compounds which, by topical application to the skin, are capable of reducing lipogenesis by the sebocytes and of consequently restricting the production of sebum.

Mention may in particular be made, among these compounds, of a coffee bean extract (FR-2 848 447) and ascorbic acid and its compounds (EP-0 771 557).

Furthermore, it is commonplace to introduce, into cosmetic products intended for caring for greasy skin, powders of natural or synthetic origin intended to absorb sebum, among which may be mentioned in particular fillers, such as polyamide (nylon) powders, talc, starch or silica. The Applicant Company has furthermore demonstrated (EP-1 493 433) that porous polyamide particles having a mean diameter, by volume, of less than or equal to 10 μm can be charged with active agents, intended in particular for caring for greasy skin, and/or with vitamin C compounds and can thus make it possible to convey these active agents into the pilosebaceous follicle onto their site of action. These compositions can comprise ascorbic acid, in addition to the charged particles. Example 7 thus discloses a water-in-oil emulsion comprising triclosan absorbed into nylon particles, and ascorbic acid.

The need remains, however, to have available compositions which make it possible to more effectively combat the aesthetic disorders of greasy skin than those provided to date in the prior art.

### SUMMARY OF THE INVENTION

The inventor has discovered that this need is met by combining, with porous particles, charged or not charged with active agent(s), an ascorbic acid compound capable of slowing down the production of sebum, provided that the composition is not a water-in-oil emulsion. The composition thus obtained makes it possible to lastingly improve the mattness of the skin. While not bound by any particular theory, this is believed to be because the ascorbic acid compound restricts the production of sebum while the porous particles make it possible to absorb the sebum at the surface of the skin and, for those which have penetrated into the pores by massaging the composition on the skin, to “suck up” the sebum at its source, indeed even to release an antiseborrhoeic active agent on its site of action. The skin is thus efficiently mattified. An antiageing cream comprising an ascorbyl glucoside and nylon 6 is certainly known from Application WO 00/62789. However, aged skin is characterized, in contrast to greasy skin, by a reduction in the production of sebum, which is particularly marked after the menopause, and generally results, in “mature” or “oligoseborrhoeic” skin, in a level of sebum of less than 100 μg/cm² on the forehead.

One subject-matter of the present invention is thus the use, for the care of greasy skin, of a composition comprising, preferably in a physiologically acceptable medium, (a) at least one water-soluble ascorbic acid compound and (b) porous-polyamide particles having a mean diameter, by volume, of less than or equal to 10 μm, provided that the composition is not a water-in-oil emulsion.

It also relates to a cosmetic process for caring for greasy skin, in particular for the purpose of mattifying the skin, comprising the topical application to the latter of a composition as defined above.

The term “mattifying” is understood to mean, in the present patent application, rendering the skin visibly more matt and less shiny. The mattifying effect of the composition can be evaluated in particular using a gonioreflectometer, the ratio R between the specular reflectance and the diffuse reflectance being measured. A value of R of less than or equal to 2 generally expresses a mattifying effect.

The term “greasy skin” is understood to mean a glistening skin which exhibits in particular a level of sebum of greater than 200 μg/cm², measured on the forehead.

The term “water-soluble ascorbic acid compound” is understood to mean compounds capable of at least partially dissolving in water which can be chosen in particular from: ascorbic acid, a metal salt of phosphorylated ascorbic acid and an ascorbyl glucoside.

Mention may be made, as metal salts of phosphorylated ascorbic acid, of magnesium ascorbyl phosphate and sodium ascorbyl phosphate.

The term “ascorbyl glucoside” is understood to mean the condensation product of glucose, in the D form, that is to say in the α- or β-glucopyranose or α- or β-furanose form, or in the L form, with ascorbic acid, preferably in the L form. L-Ascorbic acid 2-O-α-D-glucopyranoside is preferred for use in the present invention. It is available in particular from Hayashibara.

The amount of water-soluble ascorbic acid compound, e.g., ascorbyl glucoside, used according to the invention is not particularly limited but preferably can represent from 0.01 to 10% and more preferably from 0.01 to 5% of the total weight of the composition.

The expression “porous particles” denotes particles having a structure comprising pores. Although this embodiment is only optional, this porous structure can make possible, at least in part, the incorporation of one or more active agents in the particles. Within the meaning of the present invention, the expression “charged particles” will be used below to denote the particles according to the invention which include an active agent.

The structure of the particles can be of matrix type, such as a sponge. It can also be of vesicular type, that is to say that the particle exhibits an internal cavity delimited by a porous wall. The porosity associated with the size of the particles is characterized quantitatively by their specific surface.

The porous particles of the invention preferably exhibit a specific surface, measured according to the BET method, of greater than or equal to 1 m²/g. The BET (Brunauer-Emmet-Teller) method is a method well known to a person skilled in the art. It is described in particular in “The Journal of the American Chemical Society”, vol. 60, page 309, February 1938, and corresponds to the international standard ISO 5794/1 (Annex D). The specific surface measured according to the BET method corresponds to the total specific surface, that is to say that it includes the surface formed by the pores.

According to a preferred embodiment, the particles of the invention exhibit a specific surface, measured by BET, ranging in particular from 2.5 to 100 m²/g.

The porous particles according to the invention are generally distinct particles. The expression “distinct particles” denotes particles which are not grouped together in the aggregate or agglomerate form. These particles exhibit in particular a density of greater than or equal to 0.15 g/cm³ and ranging in particular from 0.2 to 5 g/cm³.

As mentioned above, the particles according to the present invention preferably have a volume-average diameter of less than or equal to 10 μm. This is believed to be because such particles can enter the sebaceous follicle by application of a mechanical force. This mechanical force generally originates from a massaging, which, in addition to the pressure which it exerts, generates a sucking effect in the follicle. The particles thus gradually arrive at the follicle channel, in which they are capable of absorbing the sebum and, if appropriate, of releasing the active compound which they carry. The constituent material of the particles is subsequently discharged by virtue of the flow of sebum and/or of the growth of the hair, thus making it possible to avoid any possible undesirable reaction of the body with regard to this material.

It should be noted that particles having a diameter of greater than 10 μm, even with application of a similar mechanical force, for the most part remain located on the surface of the skin without penetrating therein.

According to a specific embodiment of the invention, the particles have a volume-average diameter of greater than or equal to 0.1 μm and in particular ranging from 0.5 to 8 μm. The particles used according to the invention are preferably porous particles, in particular spherical particles, with a number-average diameter which can range for example from 0.1 to 50 μm, in particular from 0.1 to 20 μm and very particularly from 0.5 to 10 μm.

According to an alternative form of the invention, the particles are characterized by their homogeneity with regard to particle size. In particular, they preferably exhibit a polydispersity index, PI, ranging from 1 to 4 and in particular of less than or equal to 3. This polydispersity index is defined as the ratio D(4,3)/D(3,2), in which D(4,3) denotes the volume-average diameter and D(3,2) denotes the surface-average diameter. These two values are commonly measured using laser diffraction particle sizers, such as that sold under the name “Mastersizer 2000” by Malvern.

The porous particles of the invention can have varied shapes, in particular a globular shape and especially a substantially spherical shape.

Preferably, use is made, as porous particles according to the invention, of polyamide particles, in particular particles of nylon 6, nylon 6,6, nylon 12 or nylon 6,12, such as those sold by Atofina under the generic name “Orgasol”.

Among these compounds, preference is given very particularly to nylon 12.

The porous particles used according to the invention may be charged with at least one care active agent for greasy skin or may be uncharged (empty).

When charged particles are used according to the invention, these can be prepared according to conventional methods, in particular by impregnation. In particular, the charged particles used according to the invention can be obtained by impregnation of preformed porous particles with at least one active compound. Advantageously, this protocol does not require the presence of a pore-forming agent.

By way of example, the porous particles including the active agent are capable of being obtained according to an impregnation process comprising the following stages:
the dissolution of the active agent to be encapsulated in a solvent, such as acetone or ethanol, in order to obtain an impregnation solution,
the impregnation of the porous particles by the impregnation solution,
the evaporation of the solvent until a dry powder is obtained.

The powder thus obtained generally comprises only a very small proportion of residual solvent, of the order of 1/10 ppm.

Mention may in particular be made, as solvents which can be used in such an impregnation process, of acetone, ethanol, isopropanol, dichloromethane or ethyl acetate. Of course, the choice of the solvent is made taking into account the nature of the compounds to be encapsulated.

Generally, the composition comprises from 0.1 to 50% by weight, preferably from 0.1 to 20% by weight and more preferably from 0.5 to 5% by weight of porous particles (charged or uncharged), with respect to the total weight of the composition.

When these particles are charged, they preferably comprise a care active agent for greasy skin.

The expression “care active agent for greasy skin” is understood to mean, in the context of the present invention, a compound which has by itself, that is to say not requiring the involvement of an external agent in order to activate it, a biological activity which can in particular be:
a desquamating activity (which makes possible the opening of the blackheads), and/or
an antimicrobial activity (in particular with regard to P. acnes), and/or
an antiinflammatory activity, and/or
a sebum-regulating activity, and/or
an antioxidizing activity (which prevents oxidation of squalene and the formation of blackheads) and/or mixtures thereof.

The care active agent for greasy skin can thus be chosen from: desquamating and/or antimicrobial and/or antiinflammatory and/or sebum-regulating and/or antioxidizing agents and/or mixtures thereof.

1. Desquamating Agents

The term “desquamating agent” is understood to mean any compound capable of acting:
either directly on desquamation by promoting exfoliation, such as β-hydroxy acids, in particular salicylic acid and its compounds (including 5-(n-octanoyl)salicylic acid); α-hydroxy acids, such as glycolic acid, citric acid, lactic acid, tartaric acid, malic acid or mandelic acid; urea; gentisic acid; oligofucoses; cinnamic acid; Saphora japonica extract; resveratrol and some jasmonic acid compounds;
or on the enzymes involved in desquamation or decomposition of the corneodesmosomes, such as glycosidases, stratum corneum chymotryptic enzyme (SCCE) or indeed even other proteases (trypsin, chymotrypsin-like). Mention may be made of amino-sulphonic compounds and in particular N-(2-hydroxy-ethyl)piperazine-N′-2-ethanesulphonic acid (HEPES); 2-oxothiazolidine-4-carboxylic acid (procysteine) compounds; compounds of α-amino acids of glycine type (as disclosed in EP-0 852 949, and the sodium methylglycinediacetate sold by BASF under the trade name Trilon M); honey; or sugar compounds, such as O-octanoyl-6-D-maltose and N-acetylglucosamine.

5-(n-Octanoyl)salicylic acid is preferred for use in the present invention.

2. Antimicrobial Agents

The antimicrobial agents capable of being used in the composition according to the invention include, in particular, 2,4,4′-trichloro-2′-hydroxydiphenyl ether (or triclosan), 3,4,4′-trichloro-carbanilide, phenoxyethanol, phenoxypropanol, phenoxy-isopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopirox olamine, undecylenic acid and its salts, benzoyl peroxide, 3-hydroxybenzoic acid, 4-hydroxy-benzoic acid, phytic acid, N-acetyl-L-cysteine, lipoic acid, azelaic acid and its salts, arachidonic acid, resorcinol, octopirox or piroctone olamine, octoxyglycerin, octanoylglycine, caprylyl glycol, 10-hydroxy-2-decanoic acid, the dichlorophenyl imidazole dioxolane and its compounds disclosed in Patent WO 93/18743, copper pidolate, salicylic acid, iodopropynyl butylcarbamate, farnesol, phyto-sphingosines and their mixtures.

Preferred antimicrobial agents include octopirox, salicylic acid and iodopropynyl butylcarbamate.

3. Antiinflammatory Agents

Useful antiinflammatory or soothing agents which can be used in the composition according to the invention include: pentacyclic triterpenes and plant extracts (for example: Glycyrrhiza glabra) comprising them, such as β-glycyrrhetinic acid and its salts and/or its compounds (glycyrrhetinic acid monoglucuronide, stearyl glycyrrhetinate or 3-stearoyloxyglycyrrhetinic acid), ursolic acid and its salts, oleanolic acid and its salts, betulinic acid and its salts, bisabolol, an extract of Paeonia suffruticosa and/or lactiflora, salts of salicylic acid and in particular zinc salicylate, phycosaccharides from Codif, an extract of Laminaria saccharina, canola oil, extracts of camomile, allantoin, Sepivital EPC (phosphoric diester of vitamin E and C) from Seppic, omega-3 unsaturated oils, such as musk rose, blackcurrant seed, echium or fish oils, extracts of plankton, capryloyl glycine, Seppicalm VG (sodium palmitoylproline and Nymphea alba) from Seppic, an extract of Pygeum, an extract of Boswellia serrata, an extract of Centipeda cunnighami, an extract of Helianthus annuus, an extract of Linum usitatissimum, tocotrienols, extracts of Cola nitida, extracts of Centella asiatica, piperonal, an extract of clove, an extract of Epilobium angustifolium, aloe vera, an extract of Bacopa monieri, phytosterols, niacinamide, cortisone, hydrocortisone, indomethacin, betamethasone and their mixtures.

The anti-inflammatory agents preferred for use in the present invention are extracts of Centella asiatica, β-glycyrrhetinic acid and its salts, α-bisabolol and niacinamide.

4. Sebum-Regulating Agents

When the composition according to the invention comprises a sebum-regulating agent, such as a 5α-reductase inhibitor, the latter can be chosen in particular from:
retinoids and in particular retinol;
sulphur and sulphur compounds;
zinc salts, such as zinc lactate, gluconate, pidolate, carboxylate, salicylate and/or cysteate;
selenium chloride;
vitamin B6 or pyridoxine;
the mixture of capryloyl glycine, of sarcosine and of extract of Cinnamomum zeylanicum sold in particular by Seppic under the trade name Sepicontrol A5®;
an extract of Laminaria saccharina, sold in particular by Secma under the trade name Phlorogine®;
an extract of Spiraea ulmaria, sold in particular by Silab under the trade name Sebonormine®;
plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by Maruzen;
an extract of Serenoa repens sold in particular by Euromed;
plant extracts of the genus Silybum;
plant extracts comprising sapogenins and in particular extracts of Dioscorea species rich in diosgenin; and
extracts of Eugenia caryophyllata comprising eugenol or eugenyl glucoside.

Mixtures may be used.

Zinc salts are preferred for use in the present invention.

5. Antioxidizing Agents

The antioxidizing agents preferred for use in the present invention include tocopherol and its esters, such as tocopherol acetate, BHT and BHA.

The active agent or agents used in the composition according to the invention can represent for example from 1 to 50%, preferably from 2 to 40% and better still from 5 to 30% of the total weight of the charged particles.

The composition according to the invention is preferably generally suitable for topical application to the skin, generally the skin of the face, and thus generally comprises a physiologically acceptable medium, that is to say a medium compatible with the skin and/or its superficial body growths. It is preferably a cosmetically acceptable medium, that is to say a medium which exhibits a pleasant colour, a pleasant smell and a pleasant feel and which does not bring about unacceptable discomfort (smarting, stabbing pains, redness) likely to dissuade the consumer from using this composition.

This composition can be used as a care composition, that is to say a leave-on composition, or as a cleaning/make-up-removing composition, that is to say a rinse-off composition.

The composition according to the invention can be provided in any form, including any pharmaceutical dosage form conventionally used for topical application and in particular in the form of dispersions of the lotion or gel type, of emulsions with a liquid or semiliquid consistency of the milk type, obtained by dispersion of a fatty phase in an aqueous phase (O/W), of suspensions or emulsions with a soft, semi-solid or solid consistency of the cream or gel type, of multiple (W/O/W or O/W/O) emulsions, of microemulsions, of vesicular dispersions of ionic and/or nonionic type, or of wax/aqueous phase dispersions. These compositions are prepared according to the normal methods. Preferably, the composition is not a water-in-oil emulsion.

According to a preferred embodiment, the composition used according to the invention includes water and it is advantageous to provide it in the form of an O/W emulsion. This is because the composition is preferably applied to persons exhibiting greasy skin and it is therefore desirable for it to be formulated so that its texture is as light as possible and as nongreasy as possible.

In the case where it is found in the form of an O/W emulsion, the composition according to the invention can comprise, for example, as surfactants, at least one compound chosen from: esters of polyols and of a fatty acid with a saturated or unsaturated chain comprising, for example, from 8 to 24 carbon atoms and better still from 12 to 22 carbon atoms, and their oxyalkylenated compounds, that is to say comprising oxyethylene and/or oxypropylene units, such as esters of glycerol and of a C₈-C₂₄ fatty acid, and their oxyalkylenated compounds, esters of sorbitol and of a C₈-C₂₄ fatty acid, and their oxyalkylenated compounds, or esters of a sugar (sucrose, glucose, alkylglucose) and of a C₈-C₂₄ fatty acid, and their oxyalkylenated compounds; esters of polyethylene glycol and of a C₈-C₂₄ fatty acid, and their oxyalkylenated compounds; ethers of polyalkylene glycol and of C₈-C₂₄ fatty alcohols; ethers of a sugar and of C₈-C₂₄ fatty alcohols, and their mixtures.

In an alternative form, the composition according to the invention in the emulsion form can include an ionic amphiphilic polymer and be devoid of emulsifier.

This composition can additionally comprise various adjuvants commonly used in the cosmetics field, such as polyols; silicone oils, which may be volatile or nonvolatile, and/or hydrocarbon oils and/or fatty acid esters; fatty alcohols; fillers; preservatives; sequestering agents; colorants; fragrances; thickening and gelling agents, in particular polysaccharides, such as xanthan gum, acrylamide homo- and copolymers, acrylamidomethylpropanesulphonic acid (AMPS) homo- and copolymers and acrylic homo- and copolymers which are optionally crosslinked; and pH adjusters.

Of course, a person skilled in the art will take care to choose this or these optional additional compounds and/or their amounts so that the advantageous properties of the composition according to the invention are not, or not substantially, detrimentally affected by the envisaged addition.

Mention may be made, as fillers, for example, of polyamide fibres, microspheres based on acrylic copolymers such as those made of ethylene glycol dimethacrylate/lauryl methacrylate copolymer sold by Dow Corning under the name of Polytrap; poly(methyl methacrylate) microspheres, sold under the name Microsphere M-100 by Matsumoto or under the name Covabead LH85 by Wackherr; expanded powders, such as hollow microspheres and in particular the microspheres formed of a terpolymer of vinylidene chloride, of acrylonitrile and of methacrylate sold under the name Expancel by Kemanord Plast under the references 551 DE 12 (particle size of approximately 12 μm and density of 40 kg/m³), 551 DE 20 (particle size of approximately. 30 μm and density of 65 kg/m³) and 551 DE 50 (particle size of approximately 40 μm) or the microspheres sold under the name Micropearl F 80 ED by Matsumoto; powders formed of natural organic materials, such as starch powders, in particular powders formed of maize, wheat or rice starches which may or may not be crosslinked, such as the powders formed of starch which is crosslinked by octenylsuccinic anhydride sold under the name Dry-Flo by National Starch; silica; metal oxides, such as titanium dioxide or zinc oxide; mica; and their mixtures.

The amount of filler(s) can range, for example, from 0.05 to 20% by weight and better still from 0.1 to 10% by weight, with respect to the total weight of the composition.

The composition used according to the invention can also comprise, in addition to the porous particles (charged or uncharged) and the ascorbic acid compound, care active agents for greasy skin chosen from the lists indicated above which can be identical to or different from the active agents optionally included in the charged particles. They can in particular be salicylic acid and its compounds.

The composition according to the invention preferably generally has a pH ranging from 3 to 9, preferably from 4 to 7 and better still from 4 to 6.

It is generally applied to the skin of the face and optionally of the neck by gentle massaging.

The invention will now be illustrated by the following nonlimiting examples. In these examples, the amounts are shown as percentage by weight.

### EXAMPLES

### Example 1

### Fluid for Greasy Skin Comprising Uncharged Particles

An O/W emulsion having the composition below is prepared in a way conventional to a person skilled in the art.

Phase A

| | |
|---|---|
| Water | 61% |
| Glycerol | 5% |
| Preservatives | 0.5% |

Phase B

| | |
|---|---|
| Mixture of glyceryl stearate and of | 2% |
| polyoxyethylene (100 EO) stearate | |
| Cetyl alcohol | 1% |
| Oxyethylenated (20 EO) sorbitan monolaurate | 2% |
| Preservatives | 0.2% |
| Isononyl isononanoate | 6% |
| Octyldodecanol | 4% |
| 5-(n-Octanoyl)salicylic acid | 0.5% |

Phase C

| | |
|---|---|
| Cyclohexasiloxane | 5% |
| Xanthan gum | 0.2% |
| Crosslinked acrylic copolymer | 0.45% |

Phase D

| | |
|---|---|
| Water | q.s. for 100% |
| Sodium hydroxide | q.s. for pH 5 |

Phase E

| | |
|---|---|
| Water | 3% |
| L-Ascorbic acid 2-O-α-D-glucopyranoside | 0.5% |
| Citric acid | 0.17% |

Phase F

| | |
|---|---|
| Nylon 12 particles | 3% |

Phase G

| | |
|---|---|
| Starch | 3% |

Procedure: Phase A is heated with stirring at 80° C. until completely dissolved and then the temperature is brought back to 70° C. Phase B is heated with stirring at 70° C. until a clear phase is obtained and is then added to phase A with stirring. The constituents of phase C are mixed at ambient temperature and then phase C is added to the mixture of phases A and B. The constituents of phase D are mixed at ambient temperature and are then added to the mixture of phases A, B and C. The combined product is subsequently cooled to 25° C. The constituents of phase E are mixed at ambient temperature and then phase E is added to the mixture obtained above. Phases F and G are successively dispersed in this mixture.

### Example 2

### Fluid for Greasy Skin Comprising Charged Particles

a) Preparation of the Charged Porous Particles

0.5 g of tocopherol and 1 g of 5-(n-octanoyl)salicylic acid are dissolved in acetone. 13.5 g of porous nylon 12 particles, sold under the name “Orgasol 2002 UD NAT COS” by Atofina, are introduced into this mixture. The dispersion is subsequently introduced into a rotary evaporator in order to remove the acetone. A powder charged with tocopherol and salicylic acid compound is then obtained.

b) Preparation of the Cosmetic Composition

An O/W emulsion having the composition below is prepared in a way conventional to a person skilled in the art.

| | | |
|---|---|---|
| A | Water | 61% |
| | Glycerol | 5% |
| | Preservatives | 0.5% |
| B | Glyceryl and polyoxyethylene (100 EO) | 2% |
| | stearate | |
| | Cetyl alcohol | 1% |
| | Polysorbate 20 | 2% |
| | Preservatives | 0.2% |
| | Isononyl isononanoate | 6% |
| | Octyldodecanol | 4% |
| | 5-(n-Octanoyl)salicylic acid | 0.5% |
| C | Cyclohexasiloxane | 5% |
| | Xanthan gum | 0.2% |
| | Acrylic copolymer (Pemulen TR2 from Noveon) | 0.45% |
| D | Water | q.s. for 100% |
| | Sodium hydroxide | q.s. for pH 5 |
| E | Water | 3% |
| | Magnesium ascorbyl phosphate | 0.3% |
| | Citric acid | 0.17% |
| F | Charged nylon particles | 3% |

Procedure

Phase A is heated with stirring at 80° C. until completely dissolved and then the temperature is brought back to 70° C. Phase B is heated with stirring at 70° C. until a clear phase is obtained and is then added to phase A with stirring. The xanthan gum and the acrylic copolymer are dispersed in the cyclohexa-siloxane at ambient temperature for 10 minutes and phase C is added to the mixture A+B. Phase D, dissolved beforehand, is then added to the mixture thus obtained. The mixture A+B+C+D is subsequently cooled to 25° C. The constituents of phase E are mixed at ambient temperature and then phase E is added to the mixture obtained above. Phase F is subsequently added.

This composition can be applied to the face in the morning and/or evening in order to mattify greasy skin with a tendency towards acne.

### Example 3

### Evaluation of the Effectiveness of the Composition According to the Invention

The in vivo effectiveness of two fluids having the compositions indicated in Table 1 below was compared.

TABLE 1

| | Composition A | |
|---|---|---|
| | according to the | Control |
| Ingredient | invention | Composition B |
| L-Ascorbic acid 2- | 0.2% | 0% |
| O-α-D-glucopyranoside | | |
| Zinc salt | 0.1% | 0.002% |
| Tocopherol | 0.03% | 0% |
| Other active | 0% | 0.16% |
| agents* | | |
| Nylon 12 particles | 0.9% | 0% |
| Nylon 6,6 fibres | 3% | 2% |
| Hydroxy acids | 0.67% | 0.33% |
| Fillers | 4% | 8% |
| Emollients/fatty | 16% | 2% |
| substances | | |
| Polyols | 7% | 5% |
| pH adjusters | 0.22% | 1.4% |
| Alcohol | 0% | 25% |
| Preservatives | 1.2% | 0.02% |
| Thickening/gelling | 0.55% | 1.4% |
| agents | | |
| Surfactants | 5% | 0% |
| Fragrance | 0.4% | 0.5% |
| Water | q.s. for 100% | q.s. for 100% |

*having no-promoting effect on the production of sebum

These compositions were tested on male or female subjects aged from 18 to 35 having greasy skin (Sebutape score>2). Compositions A and B were applied for 4 weeks at the rate of two applications daily.

The clinical scoring of the pores was carried out with a Dermascore before treatment and after 4 weeks of treatment. Sebutapes were also removed and subjected to image analysis.

Results:

For the subjects who had applied Composition A, the score after 4 weeks of treatment was 1.93±1.03 (p<0.001), whereas this score was 2.88±1.08 before treatment, i.e. an improvement of 33.1%. The texture of the skin is significantly improved and the pores are less visible. In contrast, no significant improvement in the narrowing of the pores is recorded for the subjects who had applied Composition B.

In addition, for the subjects who had applied Composition A, the image analysis of the Sebutapes showed a reduction of 77.8% in the number of active sebaceous glands after 4 weeks of treatment, with respect to the number of glands present before treatment. At the same time, the total area occupied by the sebaceous glands was reduced by 83.8% and the degree of occupation of the sebaceous glands was reduced by 83.7%. These results thus show a significant reduction in sebaceous secretion after application of Composition A. The number of sebaceous glands was reduced by only 11.8% for the subjects who had applied Composition B.

Thus, Composition A, which differs essentially from Composition B only in the presence of an ascorbyl glucoside and of polyamide particles, markedly reduces sebaceous secretion with respect to Composition B.

### Example 4

### Demonstration of the Sebum-Moderating Activity of Various Ascorbic Acid Compounds

Various ascorbic acid compounds were tested on a model of cultured immortalized human sebocytes resulting from the SZ95 line described in Zouboulis, C. C., Seltmann, H., Neitzel, H. and Orfanos, C. E., Establishment and Characterization of an Immortalized Human Sebaceous Gland Cell Line, J. Invest. Dermatol., 113, 1011-1020 (1999).

These compounds were composed of L-ascorbic acid 2-O-α-D-glucopyranoside (hereinafter ascorbyl glucoside), of ascorbic acid, of ascorbyl palmitate and of magnesium ascorbyl phosphate.

The test consisted in measuring the amount of lipids produced by the sebocytes of the line (at confluence) in the presence or absence of active agents diluted in the culture medium. After 2 days of treatment, the adherent cells are treated with Nile Red (1 μg/ml). The content of lipids is subsequently quantified by measuring the fluorescence of the dye (two excitation/emission pairs: 485-540 nm for the neutral lipids and 540-620 nm for the nonneutral lipids). The results are given for the neutral lipids (components of the sebum).

The experiment is carried out in octuplicate (tested and control products) in a 96-well plate.

The results are presented in Table 2 below.

TABLE 2
Inhibition of lipogenesis by ascorbic acid compounds

| | Effectiveness (% with | | |
|---|---|---|---|
| | respect to the control) | | |
| | 0.04% | 0.004% | 0.0004% |
| Ascorbic acid | 73 | 84 | ns |
| Ascorbyl glucoside | 91 | 84 | 89 |
| Magnesium ascorbyl | 80 | 90 | ns |
| phosphate | | | |
| Ascorbyl palmitate | — | 174 | 143 |

ns: not significant

As emerges from this table, ascorbyl glucoside reduces lipogenesis at an effective concentration which is ten times lower than that necessary for ascorbic acid to produce a comparable level of inhibition. In addition, ascorbyl palmitate has a stimulating (and not inhibiting) effect on lipogenesis, rendering it unsuitable for use in the care of greasy skin.

The above written description of the invention provides a manner and process of making and using it such that any person skilled in this art is enabled to make and use the same, this enablement being provided in particular for the subject matter of the appended claims, which make up a part of the original description and including the use, for example the cosmetic use, for the care of greasy skin, of a composition including, in a physiologically acceptable medium, (a) at least one water-soluble ascorbic acid compound and (b) porous polyamide particles having a mean diameter, by volume, of less than or equal to 10 μm, provided that the composition is not a water-in-oil emulsion.

As used above, the phrases “selected from the group consisting of,” “chosen from,” and the like include mixtures of the specified materials.

As used herein, all ingredient classes can be constituted by a mixture of such ingredients. For example, the invention composiotn can comprise several ascorbic acid compounds and different types of porous polyamide particles, e.g., charged and uncharged.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s), “including,” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for the treatment of greasy skin, comprising applying to skin in need of such treatment a composition comprising, in a physiologically acceptable medium, (a) at least one water-soluble ascorbic acid compound and (b) porous polyamide particles having a mean diameter, by volume, of less than or equal to 10 μm, provided that the composition is not a water-in-oil emulsion.

2. The method according to claim 1, wherein the ascorbic acid compound is selected from the group consisting of ascorbic acid, a metal salt of phosphorylated ascorbic acid, an ascorbyl glucoside, and mixtures thereof.

3. The method according to claim 1, wherein the ascorbic acid compound is selected from the group consisting of magnesium ascorbyl phosphate, sodium ascorbyl phosphate, and mixtures thereof.

4. The method according to claim 1, wherein said compostion comprises L-ascorbic acid 2-O-α-D-glucopyranoside.

5. The method according to claim 1, wherein the porous particles have a specific surface, measured according to the BET method, of greater than or equal to 1 m²/g.

6. The method according to claim 1, wherein the porous particles have a volume-average diameter ranging from 0.5 to 8 μm.

7. The method according to claim 1, wherein the porous particles are particles selected from the group consisting of nylon 6, nylon 6,6, nylon 12 and nylon 6,12 particles, and mixtures thereof.

8. The method according to claim 1, wherein the porous particles are charged with at least one care active agent for greasy skin.

9. The method according to claim 8, wherein the porous particles are obtained according to an impregnation process comprising:
dissolving the active agent to be charged in a solvent in order to obtain an impregnation solution,
impregnating the porous particles with the impregnation solution,
evaporating the solvent of the impregnation solution until a dry powder is obtained.

10. The method according to claim 1, wherein the composition comprises from 0.5 to 5% by weight of porous particles, with respect to the total weight of the composition.

11. The method according to claim 8, wherein the care active agent for greasy skin is selected from the group consisting of desquamating, antimicrobial, anti-inflammatory, sebum-regulating, and antioxidizing agents, and mixtures thereof.

12. The method according to claim 8, wherein the porous particles are charged with 5-(n-octanoyl)-salicylic acid.

13. The method according to claim 8, wherein the porous particles are charged with an antimicrobial agent chosen from octopirox, salicylic acid, iodopropynyl butylcarbamate, and mixtures thereof.

14. The method according to claim 8, wherein the porous particles are charged with an antiinflammatory agent chosen from extracts of Centella asiatica, β-glycyrrhetinic acid and its salts, α-bisabolol, niacinamide, and mixtures thereof.

15. The method according to claim 8, wherein the porous particles are charged with chosen from zinc salts and their mixtures.

16. The method according to claim 8, wherein the porous particles are charged with an antioxidizing agent chosen from tocopherol, tocopherol acetate, and mixtures thereof.

17. The method according to claim 8, wherein the active agent represents from 5 to 30% of the total weight of the charged particles.

18. The method according to claim 1, wherein the composition is in the form of an O/W emulsion.

19. The method according to claim 1, wherein the composition has a pH ranging from 4 to 6.

20. The method according to claim 1, wherein said skin exhibits a level of sebum of greater than 200 μg/cm², and wherein after application the ratio R between the specular reflectance and the diffuse reflectance of the treated skin provides a value of R of less than or equal to 2.
